(19) United States
(12) Patent Application Publication
(10) Pub. No.: US 2011/0165607 A1
(43) Pub. Date: Jul. 7, 2011

(54) METHOD FOR EVALUATING STATUS OF SKIN BARRIER FUNCTION OF NATURAL MOISTURIZING FACTOR USING BLEOMYCIN HYDROLASE ACTIVITY AS INDICATOR

(75) Inventors: Atsushi Takeda, Kanagawa (JP); Toshihiko Hibino, Kanagawa (JP)

(73) Assignee: Shiseido Company, Ltd.

(21) Appl. No.: 12/736,918

(22) PCT Filed: May 21, 2009
(86) PCT No.: PCT/JP2009/059362

§ 371 Date: Feb. 8, 2011

(30) Foreign Application Priority Data
May 23, 2008 (JP) 2008-135944

(51) Int. Cl. C12Q 1/37

(52) U.S. Cl. 435/23

(57) ABSTRACT

The present invention provides a method for evaluating the status of the skin barrier function of natural moisturizing factor (NMF) using the activity of bleomycin hydrolase in skin tissue as an indicator.

### TECHNICAL FIELD

The present invention relates to a method for evaluating the status of the skin barrier function of natural moisturizing factor (NMF) and screening skin barrier function improvers using the activity of bleomycin hydrolase in skin tissue as an indicator, and to a method for improving the skin barrier function of NMF by increasing the activity of bleomycin hydrolase in skin tissue.

### BACKGROUND ART

Keratin fibers of the granular layer of the skin bind to and aggregate in protein referred to as filaggrin during keratinization, and form a unique pattern referred to as a “keratin pattern”. Although profilaggrin (consisting of a linear arrangement of 10 to 12 filaggrin units), which is a precursor of filaggrin, is present in large amounts in keratohyalin granules within granulocytes, together with the formation of filaggrin monomers, keratin fibers are made to aggregate by dephosphorylation during keratinization. Subsequently, deimination occurs due to the action of an enzyme known as peptidylarginine deiminase (PAD), and an amino acids and the like are decomposed in the superficial layer of the stratum corneum after having dissociated from keratin. These amino acids are referred to as natural moisturizing factors, and are known to play an important role in maintaining the moisture content of the stratum corneum as well as have the ability to absorb ultraviolet light (Blank, I. H., J. I. Dermatol., 18, 433 (1952); Blank, I. H., J. I. Dermatol., 21, 259 (1953)).

Ever since it was clearly demonstrated that amino acids, which are the main components of NMF, are derived from filaggrin, research has been conducted on the correlation between diseases associated with dry skin and filaggrin. In recent years, amino acids have been determined to decrease in the stratum corneum in dry skin of diseases such as senile xerosis or atopic diseases (Horii, I. et al., Br. J. Dermatol., 121, 587-592 (1989); Tanaka, M., et al., Br. J. Dermatol., 139, 618-621 (1989)).

PAD deiminizes filaggrin by acting on arginine residues thereof and converts the arginine residues to citrulline residues. Affinity between filaggrin and keratin fibers may be weakened or keratin fibers may be dissociated due to this deimination of filaggrin, thereby resulting in filaggrin being more susceptible to the action of protease and ultimately decomposed to NMF. However, which protease of the epidermis acts on the deiminized filaggrin causing it to ultimately be decomposed to NMF has not been determined. As was described at the outset, since NMF plays an important role in the moisturizing function of the skin as well as in the barrier function of the skin, it is important in terms of dermatology, cosmetology and in terms of finding a drug that improves the barrier function of the skin to determine the process that filaggrin undergoes as it is decomposed to NMF.

### PRIOR ART DOCUMENTS

### Non-Patent Documents

Non-Patent Document 1: Blank, I. H., J. I. Dermatol., 18, 433 (1952)
Non-Patent Document 2: Blank, I. H., J. I. Dermatol., 21, 259 (1953)
Non-Patent Document 3: Horii, I., Br. J. Dermatol., 121, 587-592 (1989)
Non-Patent Document 4: Tanaka, M., et al., Br. J. Dermatol., 139, 618-621 (1989)
Non-Patent Document 5: Kamata, et al., J. Biochem., 141, 69-76 (2007)

### SUMMARY OF THE INVENTION

### Problems to be Solved by the Invention

An object of the present invention is to provide a method for evaluating the status of the skin barrier function of NMF by elucidating the formation process of NMF, a method for screening drugs that improve skin barrier function, and a method for improving skin barrier function.

### Means for Solving the Problems

The inventors of the present invention conducted research for the purpose of elucidating the decomposition process of filaggrin, which is the source of NMF. First, when various enzymes were allowed to act on filaggrin deiminized with PAD, although the deiminized filaggrin did not demonstrate susceptibility to virtually any of the enzymes, it demonstrated high susceptibility to calpain-I, and was found to be decomposed into small peptide fragments. Furthermore, calpain-I demonstrated stronger differentiation activity on deiminized filaggrin than on non-deiminized filaggrin (to simply be referred to as unmodified filaggrin). In addition, calpain-I alone was unable to decompose deiminized filaggrin to amino acid units.

Moreover, when a search was made for various enzymes that decompose the small peptide fragments, these fragments were surprisingly found to be decomposed to amino acid units, namely NMF, by bleomycin hydrolase (BH). Furthermore, bleomycin hydrolase was determined to be unable to decompose deiminized filaggrin per se.

On the basis of these findings, it is thought that deiminized filaggrin in the body, from which keratin fibers are dissociated as a result of being deiminized by PAD, is first severed into somewhat smaller molecules by calpain-I, after which it is decomposed to amino acid units by bleomycin hydrolase resulting in the formation of NMF, thereby enabling demonstration of skin moisturizing function, and in turn, skin barrier function.

Thus, the present application includes the following inventions:

(1) a method for evaluating the status of the skin barrier function of natural moisturizing factor (NMF) using the activity of bleomycin hydrolase in skin tissue as an indicator;
(2) the method of (1), wherein the skin barrier function of NMF is judged to have decreased if activity of bleomycin hydrolase in the skin tissue has decreased significantly in comparison with a control skin, and the skin barrier function of NMF is judged to be normal if the activity is equal to or greater than that of the control skin;
(3) the method of (1) or (2), wherein the activity of calpain-I in skin tissue is used as an indicator;
(4) the method of (3), wherein the skin barrier function of NMF is judged to have decreased if activity of calpain-I in the skin tissue has decreased significantly in comparison with a control skin, and the skin barrier function of NMF is judged to be normal if the activity is equal to or greater than that of the control skin;
(5) a method for evaluating and screening improvers of the skin barrier function of NMF using the activity of bleomycin hydrolase in skin tissue as an indicator;
(6) the method of (5), wherein the activity of calpain-I in skin tissue is used as an indicator;
(7) a method for improving the skin barrier function of NMF by increasing the activity of bleomycin hydrolase in skin tissue; and,
(8) the method of (7), wherein the activity of calpain-I in skin tissue is also increased.

### Effects of the Invention

According to the method of the present invention, skin properties, namely the status of the skin barrier function of NMF, can be evaluated at the biochemical level.

### BRIEF DESCRIPTION OF THE DRAWINGS

FIG. 1 shows decomposition of deiminized filaggrin by various proteases.

FIG. 2 shows production of amino acids from deiminized filaggrin peptides decomposed with calpain-I by bleomycin hydrolase.

### BEST MODE FOR CARRYING OUT THE INVENTION

Bleomycin hydrolase is a cytoplasmic cysteine peptidase having a molecular weight of 250 to 280 kDa (hexamer), and its only known activity is metabolic deactivation of the glycopeptide, bleomycin, frequently used in combination chemotherapy for cancer. It includes active site residues characteristic of the papain superfamily of cysteine proteases, and the coding gene thereof is present at gene locus 17q11.2 in humans (Takeda, et al., J. Biochem., 119, 29-36, 1996). Although it is present in various tissues and its presence in the skin is also known (Kamata, et al., J. Biochem., 141, 69-76, 2007), its relationship with filaggrin has been completely unknown.

Calpain-I is also referred to as micro-calpain, and is a neutral cysteine protease activated by calcium ions. Although its function has not been adequately elucidated, it is thought to be involved in signal transduction mediated by intracellular calcium ions. Although it is also known to be present in various tissues in the same manner as bleomycin hydrolase, its relationship with filaggrin has been completely unknown.

Measurement of the bleomycin hydrolase and calpain-I relating to the present invention can be carried out quantitatively or qualitatively in accordance with any arbitrary method capable of measuring bleomycin hydrolase and calpain-I. Specific examples of such methods include various methods such as immunoassay methods that use a specific antibody to bleomycin hydrolase or calpain-I, such as ELISA using an enzyme label, RIA using a radioactive label, immunonephelometry, western blotting, latex agglutination or hemagglutination. Examples of immunoassay methodology include competitive methods and sandwich methods. In addition, measurement of bleomycin hydrolase and calpain-I can also be carried out by measuring the amount of a gene that encodes bleomycin hydrolase or calpain-I. In this case, expression of bleomycin hydrolase or calpain-I is preferably determined by measuring the amount of mRNA that encodes bleomycin hydrolase or calpain-I within cells. Extraction of mRNA and quantitative or qualitative measurement of its amount are known in the art, and can be carried out by various known methods such as PCR, 3SR, NASBA or TMA. In addition, bleomycin hydrolase and calpain-I can also be determined qualitatively using in situ hybridization or by measuring the biological activity thereof.

In the method for evaluating the status of the skin barrier function of natural moisturizing factors (NMF), the skin barrier function of NMF is judged to have decreased if the amounts of bleomycin hydrolase and calpain-I have significantly decreased in comparison with, for example a control skin, or the skin barrier function of NMF is judged to be normal if the amounts are equal to or greater than those of a control skin.

A “significant decrease in comparison with control skin” refers to the case in which the measured amount of bleomycin hydrolase or calpain-I is 80% or less, 70% or less, 60% or less, 50% or less, 30% or less or 10% or less that of a normal “control skin” judged to be normal by a physician from, for example, a dermatological standpoint. “Being equal to or greater than that of control skin” refers to the case in which the measured amount of bleomycin hydrolase or calpain-I is, for example, 80% or more, 90% or more or 100% or more that of a normal “control skin” judged to be normal by a physician from, for example, a dermatological standpoint.

In a method for evaluating and screening improvers of the skin barrier function of NMF by using the activity of bleomycin hydrolase in skin tissue as an indicator, the candidate drug is judged to an improver of the skin barrier function of NMF if the amount of bleomycin hydrolase or calpain-I in skin in which the candidate drug is allowed to act is significantly increased in comparison with, for example, a control skin on which the candidate drug has not acted. A “significant increase in comparison with a control skin” refers to the case in which the measured amount of bleomycin hydrolase or calpain-I in skin in which the candidate drug is allowed to act is, for example, 120% or more, 150% or more or 200% or more that of the “control skin”.

In a method for improving the skin barrier function of NMF by increasing the activity of bleomycin hydrolase in skin tissue, the amount of bleomycin hydrolase or calpain-I in the skin is significantly increased as compared with the amount in the skin prior to carrying out this treatment method. A “significant increase” refers to the case in which, for example, the amount of bleomycin hydrolase or calpain-I has been increased to a value of 120% or more, 150% or more or 200% or more.

Although sampling of skin stratum corneum used as a specimen can be carried out by any arbitrary method, from the viewpoint of convenience, tape stripping is carried out preferably. Tape stripping refers to a method in which a piece of adhesive tape is affixed to the skin surface, the tape is peeled off, and a stratum corneum sample is obtained by allowing the skin stratum corneum to remain adhered to the peeled adhesive tape. Use of the tape stripping method makes it possible to measure expression of bleomycin hydrolase or calpain-I simply by sampling the stratum corneum with a piece of tape, and can be used for non-invasive evaluation of chapped skin or parakeratosis by using bleomycin hydrolase or calpain-I as an indicator. A preferable method for carrying out tape stripping consists of first cleaning the surface layer of the skin with ethanol, for example, to remove any sebaceous matter, dirt and the like, gently placing a piece of adhesive tape cut to a suitable size (such as 5×5 cm) on the skin surface, pressing the adhesive tape flatly by uniformly applying pressure to the entire piece of adhesive tape, and then peeling off the adhesive tape with equal force. The adhesive tape may be commercially available cellophane tape such as Scotch Superstrength Mailing Tape (3M) or cellophane tape (Cellotape™, Nichiban).

The following provides a more detailed explanation of the present invention through specific examples thereof. Furthermore, the present invention is not limited thereto.

### Examples

The following materials were used in these experiments.

Filaggrin: Recombinant filaggrin was prepared by producing an E. coli expression system

rPAD: Recombinant PAD was produced by producing an E. coli expression system

Trypsin: Sigma

Chymotrypsin: Sigma

Cathepsin L: EMD Bioscience

Calpain I: EMD Bioscience

Cathepsin D: EMD Bioscience

Bleomycin hydrolase: Produced from newborn rat epidermis in accordance with Non-Patent Document 5

Experiment 1

In this experiment, the decomposing action of various proteases (20 types or more) on filaggrin (A) and deiminized filaggrin (B) was examined. The deiminized filaggrin was formed by completely deiminating filaggrin by reacting overnight at 37° C. with rPAD in the presence of 50 mM HEPES-NaOH buffer (pH 7.4), 50 mM DTT and 100 mM CaCl₂. The following indicates the results of decomposing filaggrin and deiminized filaggrin by typical proteases.

Filaggrin and deiminized filaggrin were respectively reacted at 37° C. with trypsin (E:S molar ratio=1:200, where E represents enzyme and S represents substrate) or chymotrypsin (E:S molar ratio=1:60) in the presence of 20 mM Tris-HCl (pH 8.0) and 20 mM CaCl₂, followed by isolating aliquots of the reaction solution over time and terminating the reaction by boiling. The gel was stained with CBB R-250 dye following SDS-PAGE.

Filaggrin and deiminized filaggrin were respectively reacted at 37° C. with cathepsin L (E:S molar ratio=1:25) in the presence of 100 mM acetate buffer (pH 5.0), 10 mM DTT and 5 mM EDTA, followed by isolating aliquots of the reaction solution over time and terminating the reaction by boiling. The gel was stained with CBB R-250 dye following SDS-PAGE.

Filaggrin and deiminized filaggrin were respectively reacted at 30° C. with calpain I (E:S molar ration=1:20) in the presence of 20 mM Tris-HCl buffer (pH 7.5), 0.5 mM CaCl₂ and 10 mM DTT, followed by isolating aliquots of the reaction solution over time and terminating the reaction by boiling. The gel was stained with CBB R-250 dye following SDS-PAGE.

Filaggrin and deiminized filaggrin were respectively reacted at 37° C. with cathepsin D (E:S molar ration=1:20) in the presence of 100 mM citrate buffer (pH 3.5), followed by isolating aliquots of the reaction solution over time and terminating the reaction by boiling. The gel was stained with CBB R-250 dye following SDS-PAGE.

Densitometric analyses of the scanned gels were carried out using an Image J computer software program with a computer installed with Windows® XP.

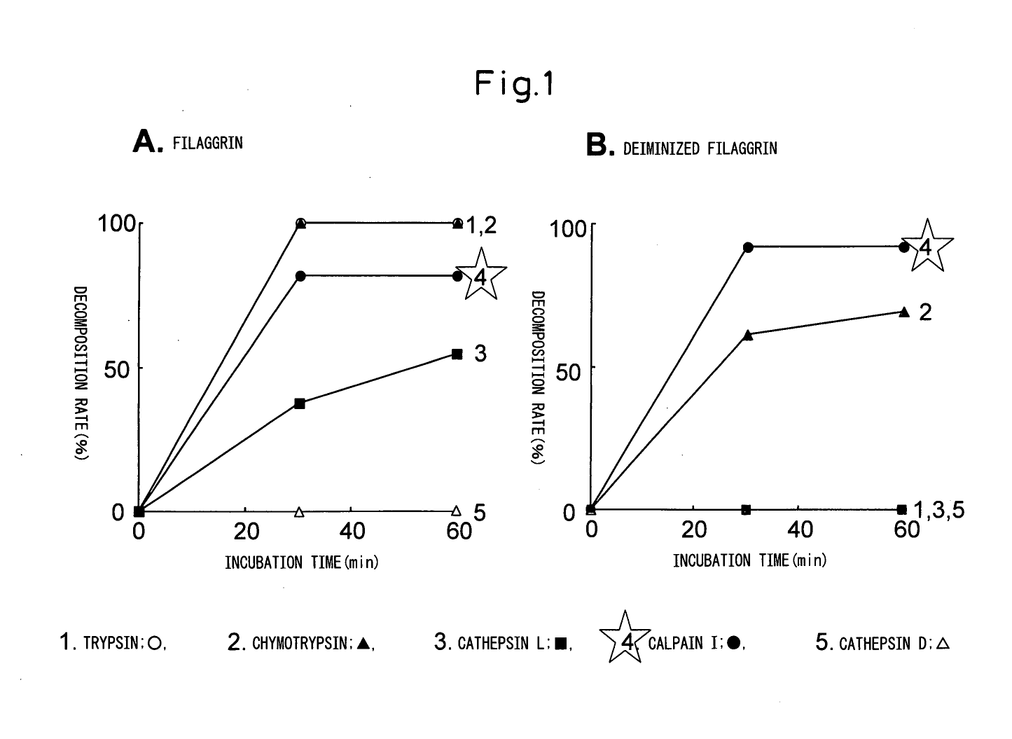

The results are shown in FIG. 1. Among the 20 or more types of enzymes investigated, calpain I demonstrated the strongest decomposing activity on deiminized filaggrin. Trypsin and cathepsin L and cathepsin D demonstrated hardly any decomposing activity on deiminized filaggrin. Furthermore, although calpain I also demonstrated decomposing activity on unmodified filaggrin, that activity was weaker than demonstrated on deiminized filaggrin.

Experiment 2

In this experiment, amino acid productivity of deiminized filaggrin peptides decomposed with calpain I by various proteases was examined. The following indicates results for the amino acid productivity of bleomycin hydrolase.
Conditions for Amino Acid Production from Deiminized Filaggrin Peptides Decomposed with Calpain I

All reactions were carried out in a HEPES buffer system since Tris buffer reacts to fluorescent reagents. α-amino groups newly formed by bleomycin hydrolase were measured with a post-label fluorescent method using fluorescamine.

Filaggrin was completely deiminized by reacting overnight at 37° C. with rPAD in the presence of 50 mM HEPES-NaOH buffer (pH 7.4), 50 mM DTT and 100 mM CaCl₂. After then reacting for 1 hour at 30° C. with calpain I, the reaction was terminated by boiling. 5 mM EDTA was then added to the peptide mixture obtained by decomposing the deiminized filaggrin and reacted at 37° C. with bleomycin hydrolase followed by isolating a portion of the reaction solution over time and terminating the reaction by boiling. A control solution was prepared by adding HEPES-NaOH buffer (pH 7.4) instead of bleomycin hydrolase, and reacted in the same manner.

After adding 100 μl of 20 mM HEPES-NaOH (pH 8.0) and 50 μl of 0.3 mg/ml fluorescamine-acetone solution to 50 μl of the reaction solution following the reaction and mixing, 500 μl of 20 mM HEPES-NaOH (pH 8.0) were further added and mixed well. Fluorescence was measured at a fixed excitation wavelength of 370 nm and fluorescence wavelength of 475 nm. The amounts of amino groups formed were estimated using the standard calibration curve for L-leucine.

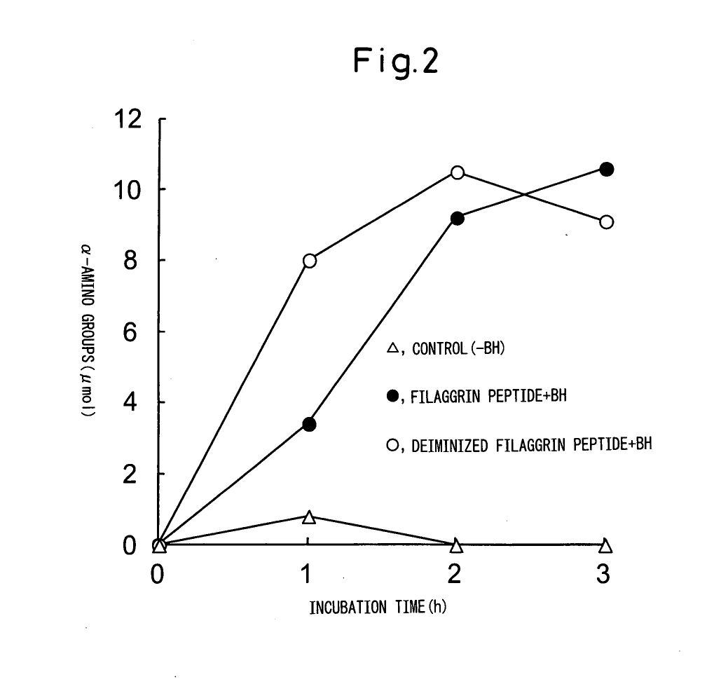

The results are shown in FIG. 2. Deiminized filaggrin peptides decomposed by calpain I was determined to rapidly produce amino acid decomposition products of bleomycin hydrolase in comparison with unmodified filaggrin peptides decomposed by calpain I.

1. A method for evaluating the status of the skin barrier function of natural moisturizing factor (NMF) using the activity of bleomycin hydrolase in skin tissue as an indicator.

2. The method according to claim 1, wherein the skin barrier function of NMF is judged to have decreased if activity of bleomycin hydrolase in the skin tissue has decreased significantly in comparison with a control skin, and the skin barrier function of NMF is judged to be normal if the activity is equal to or greater than that of the control skin.

3. The method according to claim 1, wherein the activity of calpain-I in skin tissue is used as an indicator.

4. The method according to claim 3, wherein the skin barrier function of NMF is judged to have decreased if activity of calpain-I in the skin tissue has decreased significantly in comparison with a control skin, and the skin barrier function of NMF is judged to be normal if the activity is equal to or greater than that of the control skin.

5. A method for evaluating and screening improvers of the skin barrier function of NMF using the activity of bleomycin hydrolase in skin tissue as an indicator.

6. The method according to claim 5, wherein the activity of calpain-I in skin tissue is used as an indicator.

7. A method for improving the skin barrier function of NMF by increasing the activity of bleomycin hydrolase in skin tissue.

8. The method according to claim 7, wherein the activity of calpain-I in skin tissue is also increased.

9. The method according to claim 2, wherein the activity of calpain-I in skin tissue is used as an indicator.

10. The method according to claim 9, wherein the skin barrier function of NMF is judged to have decreased if activity of calpain-I in the skin tissue has decreased significantly in comparison with a control skin, and the skin barrier function of NMF is judged to be normal if the activity is equal to or greater than that of the control skin.
